(19) United States
(12) Patent Application Publication
(10) Pub. No.: US 2015/0352370 A1
(43) Pub. Date: Dec. 10, 2015

(54) FLASHTUBE FOR LIGHT IRRADIATION TREATMENT AND PREVENTION AND LIGHT IRRADIATION TREATMENT AND PREVENTION DEVICE

(71) Applicant: PANASONIC INTELLECTUAL PROPERTY MANAGEMENT CO., LTD., Osaka-shi, Osaka (JP)

(72) Inventor: YOUJI YAMASHITA, Kyoto (JP)

(21) Appl. No.: 14/760,720

(22) PCT Filed: Dec. 16, 2013
(86) PCT No.: PCT/JP2013/007371

§ 371 Date: Jul. 14, 2015

(30) Foreign Application Priority Data
Jan. 21, 2013 (JP) 2013-008143

(51) Int. Cl. A61N 5/06

(52) U.S. Cl. CPC A61N 5/06; A61N 2005/0655; A61N 2005/0662

(57) ABSTRACT

Disclosed is a flashtube for light irradiation treatment and prevention used for medical treatment or prevention by irradiating a specific part of a living body with light, and the flashtube include a glass valve, and filler gas contained in the glass valve. The filler gas is composed of mixed gas of at least neon gas and xenon gas, and includes the xenon gas in a mixing ratio of 0.23 mol % to 1.21 mol % inclusive relative to 100 mol % of the mixed gas. Thus achieved are the flashtube for light irradiation treatment and prevention and a light irradiation treatment and prevention device capable of producing energy of the light having a wavelength ranging from 600 nm to 700 nm inclusive that are necessary for the light irradiation treatment and prevention without increasing a lighting voltage.

### TECHNICAL FIELD

The present invention relates to a flashtube for light irradiation treatment and prevention, and a light irradiation treatment and prevention device used for treating a specific part of a living body or preventing diseases.

### BACKGROUND ART

There are light irradiation treatment and prevention devices currently available for providing treatments of diseases by irradiating affected areas with light of a specific wavelength (refer to Patent Literature 1, for example). Here, the treatments include a preventive treatment performed on a healthy person to avoid contraction of diseases, and the same applies hereafter.

Light irradiation treatment and prevention devices typically use xenon discharge tubes as their light sources. However, xenon discharge tubes radiate light of a wide range of wavelengths. The light irradiation treatment and prevention devices therefore include band-pass filters (i.e., interference filters) that selectively pass only the light of wavelengths (waveband) within a specific range that is suitable for irradiating affected areas. The reason is that the light radiated from xenon discharge tubes includes light of the ultraviolet region and the infrared region. The light in the ultraviolet region has an effect on skin and the like of a human body. On the other hand, the light in the infrared range causes a thermal effect on the human body. The light irradiation treatment and prevention devices thus have band-pass filters that pass the light in a range of 566.5 nm to 780 nm inclusive. Accordingly, the light in the ultraviolet region and the infrared region is removed.

However, the light irradiation treatment and prevention devices become costly when equipped with the above-described band-pass filters because such band-pass filters are expensive. In this case, the use of such band-pass filters becomes unnecessary if the light radiated from flashtubes has wavelengths (waveband) inside the specific range suitable for the treatment and prevention.

Consequently, when a neon discharge tube filled with neon gas is used as the flashtube, for instance, it radiates light having a peak wavelength in a range between 600 nm and 700 nm inclusive. The above-described band-pass filter can hence become unnecessary. However, energy of the light radiated by the neon discharge tube is lower than energy of the light radiated by the xenon discharge tube in the wavelength range between 600 nm and 700 nm inclusive. It is therefore necessary to increase a gas pressure contained in the neon discharge tube in order to make up a deficiency in the energy of the light. There exists a disadvantage, however, that a power control circuit becomes bulky in size because a lighting voltage needs to be increased to make the neon discharge tube of high gas pressure start discharging.

### CITATION LIST

### Patent Literature

PTL 1: Unexamined Japanese Patent Publication, No. 2012-147866

### SUMMARY OF THE INVENTION

The present invention is to provide a flashtube for light irradiation treatment and prevention as well as a light irradiation treatment and prevention device capable of producing energy having wavelengths between 600 nm and 700 nm inclusive that are necessary for the light irradiation treatment and prevention without increasing a lighting voltage of the flashtube and requiring a large-sized power control circuit.

In other words, the present invention provides a flashtube for light irradiation treatment and prevention used for medical treatment or prevention by irradiating a specific part of a living body with light. The flashtube includes a glass valve, and filler gas contained in the glass valve. The filler gas is composed of mixed gas of neon gas and xenon gas, and the composition includes the xenon gas ranging from 0.23 mol % to 1.21 mol % inclusive in a ratio relative to 100 mol % of the mixed gas.

According to this composition, it becomes possible to keep the lighting voltage of 200 V or less to light the flashtube and to ensure 90% or more energy for the light radiated from the flashtube as compared to a value when the light radiated from the xenon discharge tube has 100% of energy. Thus achieved is the flashtube for light irradiation treatment and prevention that can produce energy of the wavelengths between 600 nm to 700 nm inclusive that are necessary for the light irradiation treatment and prevention without increasing the lighting voltage.

A light irradiation treatment and prevention device of the present invention is a device for use in medical treatment or prevention by irradiating a specific part of a living body with light radiated from the above-described flashtube for light irradiation treatment and prevention. The device is configured to irradiate a specific part of the living body with the light having wavelengths in a range between 600 nm and 700 nm inclusive, radiated from the flashtube for light irradiation treatment and prevention.

According to this structure, energy of the light radiated from the flashtube for light irradiation treatment and prevention can be brought to 95% or more as compared to the value when the light radiated from the xenon discharge tube has 100% of energy, without increasing the lighting voltage. This makes the expensive band-pass filter unnecessary. What can be achieved, as a result, is the light irradiation treatment and prevention device of a low cost, yet capable of radiating energy of the light having wavelength in a range between 600 nm to 700 nm inclusive that are necessary for the light irradiation treatment and prevention.

### BRIEF DESCRIPTION OF DRAWINGS

FIG. 1 is an external view of a light irradiation treatment and prevention device according to an exemplary embodiment of the present invention.

FIG. 2 is a sectional view of the light irradiation treatment and prevention device according to the exemplary embodiment.

FIG. 3 is a graph illustrating a relationship between mixing ratio of xenon gas and lighting voltage of a flashtube according to the exemplary embodiment.

FIG. 4 is a graph illustrating a relationship between the mixing ratio of xenon gas and energy of the flashtube according to the exemplary embodiment.

### DESCRIPTION OF EMBODIMENT

Description is provided hereinafter of an exemplary embodiment of the present invention with reference to the accompanying drawing. Note that the exemplary embodiment described herein is not intended to limit the scope of the present invention.

### Exemplary Embodiment

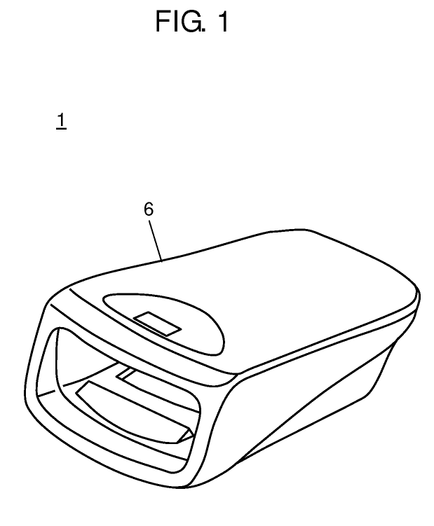

Referring to FIG. 1 to FIG. 4, description is provided hereinafter about a structure of a light irradiation treatment and prevention device equipped with a flashtube for light irradiation treatment and prevention, according to the exemplary embodiment of the present invention.

Note that the description provided here is a light irradiation device used for treating subjects of treatment stated below, as an example of the light irradiation treatment and prevention device of this exemplary embodiment. The subjects of treatment (i.e., patients), to be specific, are mainly those people who receive preventive treatment to avoid contraction of inflammatory diseases, or to relieve symptom of the diseases if contracted, or those people who receive treatment to suppress the inflammatory diseases.

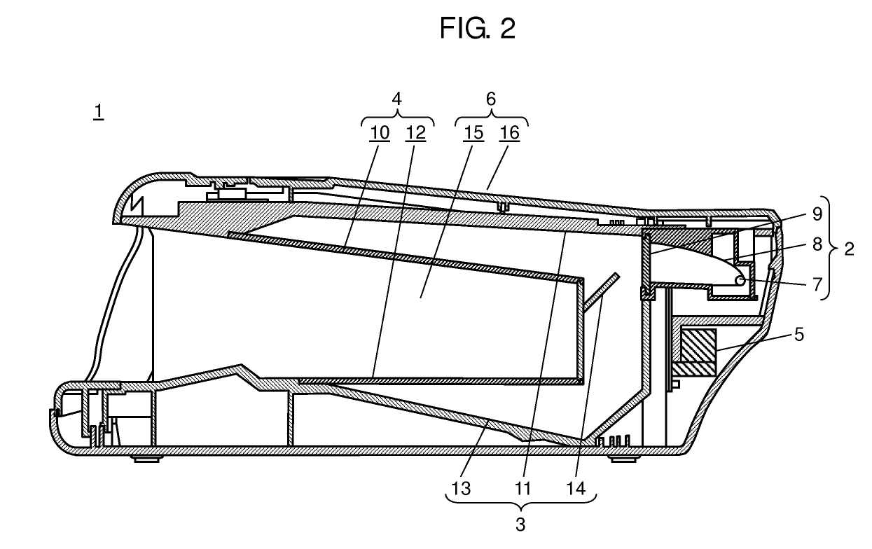

First, description is provided of a structure of the light irradiation treatment and prevention device according to this exemplary embodiment of the invention by referring to FIG. 1 and FIG. 2.

FIG. 1 is an external view of the light irradiation treatment and prevention device according to this exemplary embodiment of the invention, and FIG. 2 is a sectional view of the light irradiation treatment and prevention device according to this exemplary embodiment.

As shown in FIG. 1 and FIG. 2, light irradiation treatment and prevention device 1 of this exemplary embodiment is constructed from enclosure 6, and components including at least light-emitting unit 2, reflection frame 3, transparent frame 4, and light-emission controller 5 that are housed inside enclosure 6. Light-emitting unit 2 produces light of a wavelength necessary for the treatment or prevention. Reflection frame 3 reflects the light from light-emitting unit 2 toward a subject of irradiation. Transparent frame 4 allows the reflected light from the reflection frame 3 to pass therethrough for irradiating upon the subject of irradiation. Light-emission controller 5 controls emission of the light of light-emitting unit 2.

Light-emitting unit 2 is equipped with light source 7, reflector 8, and transparent protective panel 9 that allows the light reflected from reflector 8 to pass through, as shown in FIG. 2.

In addition, light source 7 includes a glass valve, electrodes disposed to at least one end of the glass valve, and filler gas sealed inside the glass valve. The glass valve is formed of hard glass such as silicate glass into a cylindrical shape, and both ends in a longitudinal direction of the glass valve are hermetically sealed with a part of the electrodes enclosed in it. The electrodes are formed of a metallic material such as tungsten into a rod shape, and disposed at both ends of the glass valve. In this instance, one of the electrodes constitutes a cathode electrode (i.e., negative electrode), and the other electrode constitutes an anode electrode (i.e., positive electrode).

Light source 7 serves to be a light source to irradiate a part of a user's living body where the user wants to prevent contraction of a disease or a part affected by the disease with the light, in order to suppress production of pro-inflammatory cytokine. In other words, light source 7 constitutes flashtube 7 for light irradiation treatment and prevention, of which the glass valve is filled with filler gas containing a principal component of neon gas (description is given hereafter by using the same reference marks as light source 7).

Description is provided hereinafter in more specific about the flashtube for light irradiation treatment and prevention according to this exemplary embodiment.

Flashtube 7 for light irradiation treatment and prevention has mixed gas containing at least neon gas and xenon gas as the filler gas, and it is filled inside the glass valve. Here, the filler gas preferably contains xenon gas of a ratio ranging from 0.23 mol % to 1.21 mol % inclusive relative to 100 mol % of the mixed gas. It is even more preferable that the filler gas contains xenon gas of a ratio ranging from 0.23 mol % to 0.44 mol % inclusive relative to 100 mol % of the mixed gas. The reason of this will be described in detail later. In this exemplary embodiment, all substances filled in the glass valve, regardless of their kinds and contents, are called collectively as filler gas, and these gases are expressed altogether as mixed gas when gases of many different kinds are contained.

The flashtube for light irradiation treatment and prevention is constructed as described above.

Reflector 8 is formed into a gutter shape, and it houses a part of flashtube 7 for light irradiation treatment and prevention, as shown in FIG. 2. Reflector 8 reflects the light radiated from flashtube 7 for light irradiation treatment and prevention toward the subject of irradiation who is present in a direction of an opening of the light irradiation treatment and prevention device 1.

In addition, reflection frame 3 controls an irradiation range of the light radiated from light-emitting unit 2 so as to irradiate a part of the body where the user wants to prevent contraction of disease or affected by the disease with the light. Reflection frame 3 of this exemplary embodiment includes at least first reflecting member 11, second reflecting member 13, and third reflecting member 14. First reflecting member 11 reflects the light from light-emitting unit 2 toward first transparent surface 10 of transparent frame 4. Second reflecting member 13 and third reflecting member 14 reflect the light from light-emitting unit 2 toward second transparent surface 12 of transparent frame 4.

Transparent frame 4 includes first transparent surface 10 and second transparent surface 12 that pass the light reflected from reflection frame 3. The light that passes through first transparent surface 10 of transparent frame 4 irradiates an upper area of the specific part of the living body. On the other hand, the light that passes through second transparent surface 12 of transparent frame 4 irradiates a lower area of the specific part of the living body.

Light-emission controller 5 receives setting of a light-emitting condition of flashtube 7 for light irradiation treatment and prevention. Light-emission controller 5 then controls flashtube 7 for light irradiation treatment and prevention to make it emit light based on the received light-emitting condition.

Furthermore, enclosure 6 is constructed of at least light irradiating section 15 and casing 16, as shown in FIG. 1 and FIG. 2. Light irradiating section 15 is formed from first transparent surface 10 and second transparent surface 12 that constitute transparent frame 4, and are disposed opposite to each other. Light irradiating section 15 thus irradiates a specific part of the living body inserted between first transparent surface 10 and second transparent surface 12 of transparent frame 4 with the light in both upward and downward directions, for instance. Casing 16 covers light irradiating section 15 in addition to enclosing light-emitting unit 2, reflection frame 3, transparent frame 4, light-emission controller 5, and the like.

The flashtube for light irradiation treatment and prevention, and the light irradiation treatment and prevention device of this exemplary embodiment are constructed as described above.

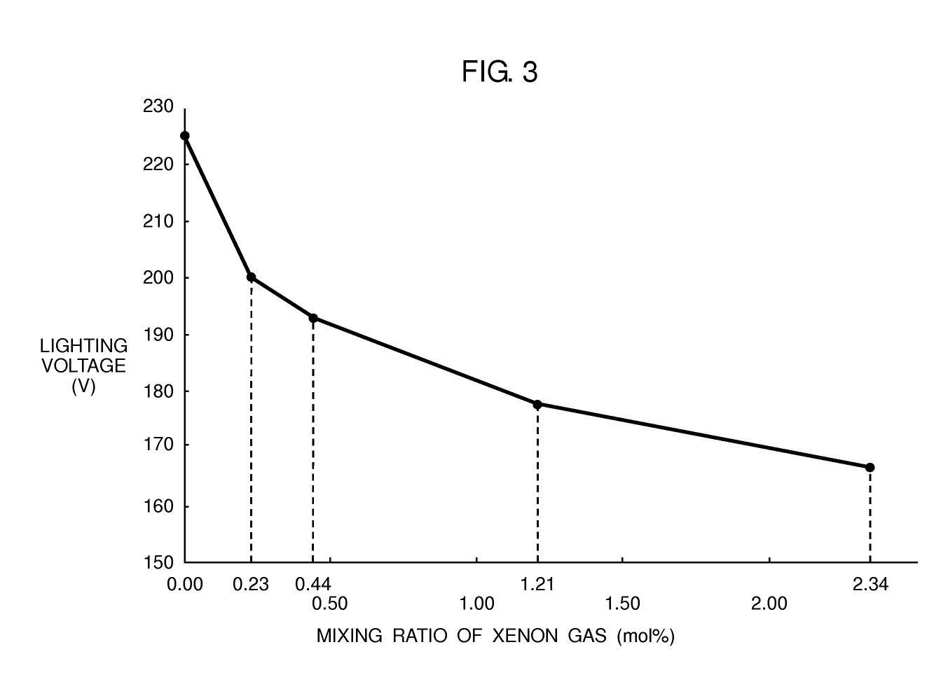

Description is provided next about a lighting voltage of the filler gas in the flashtube for light irradiation treatment and prevention of this exemplary embodiment described above with reference to FIG. 3 and Table 1.

FIG. 3 is a graph illustrating a relationship between mixing ratio of xenon gas and lighting voltage of the flashtube according to this exemplary embodiment.

Here, FIG. 3 and Table 1 show a result of measurements taken of lighting voltages of flashtube 7 for light irradiation treatment and prevention, with the mixing ratio of the mixed gas composed of neon gas and xenon gas, that is the filler gas enclosed in the glass valve, is changed. The mixing ratios of the xenon gas shown in Table 1 were measured with a quadrupole mass spectrometer.

As shown in FIG. 3 and Table 1, it is known that the lighting voltage can be lowered to 200V or less when the mixing ratio of the xenon gas is set at 0.23 mol % or more relative to 100 mol % of the mixed gas.

Described here is the reason why the lighting voltage of flashtube 7 for light irradiation treatment and prevention can be lowered.

Neon discharge tubes generally require higher lighting voltages as compared to xenon discharge tubes. When xenon gas is mixed into the neon discharge tube, the entire filler gas inside the glass valve becomes ionized easily. In other words, the lighting (or discharging) voltage of the xenon gas is generally lower between voltages of single xenon gas and single neon gas under the condition of same gas pressure. The xenon gas is therefore ionized easier than the neon gas since the electrical discharge begins upon ionization of the gas. The addition of easily ionizable xenon gas thus causes the filler gas easy to ionize. As a result, the lighting voltage of flashtube 7 for light irradiation treatment and prevention decreases.

In addition, the lighting voltage decreases further with increase in the mixing ratio of the xenon gas, as shown in FIG. 3. It is apparent, however, that a degree of the decrease in the lighting voltage becomes gentler around the point where the mixing ratio of the xenon gas exceeds 1.21 mol %. That is, it is known that contribution of the xenon gas to the decrease in the lighting voltage becomes smaller even when the xenon gas is enclosed beyond the mixing ratio of 1.21 mol %.

TABLE 1

| Mixing Ratio of Xenon Gas | Lighting Voltage |
|---|---|
| 0.00 mol % | 225.5 V |
| 0.23 mol % | 200.0 V |
| 0.44 mol % | 194.0 V |
| 1.21 mol % | 178.5 V |
| 2.34 mol % | 167.5 V |

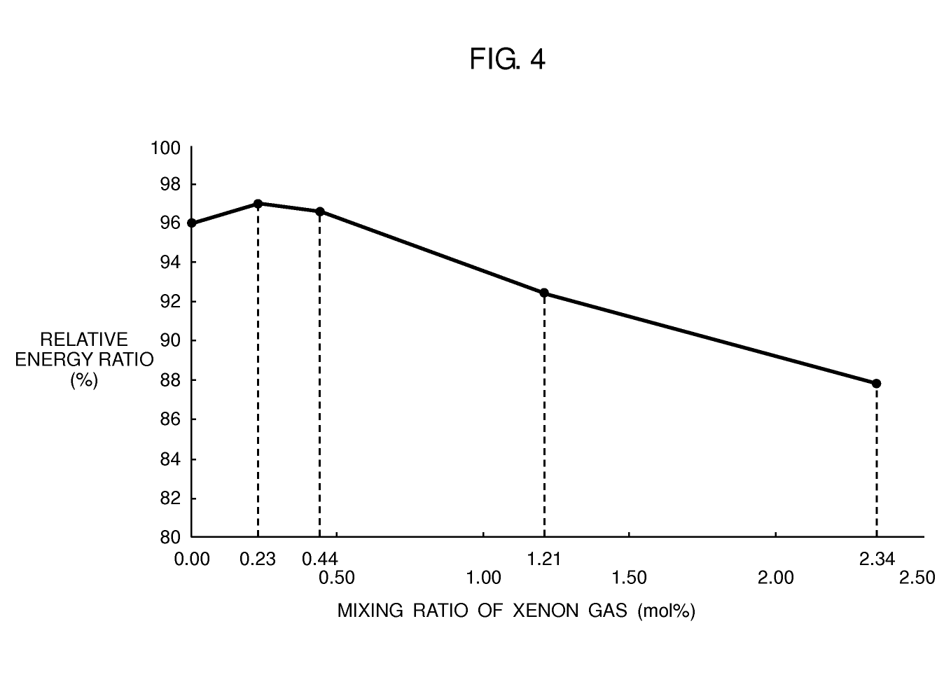

Description is provided next about energy of the light emitted from the flashtube for light irradiation treatment and prevention of this exemplary embodiment described above by referring to FIG. 4 and Table 2.

FIG. 4 is a graph illustrating a relationship between the mixing ratio of the xenon gas and energy of the light from the flashtube according to this exemplary embodiment.

FIG. 4 and Table 2 show a result of measurements taken of energy of the light in the wavelength range between 600 nm and 700 nm inclusive while changes are made in the mixing ratio of neon gas and xenon gas enclosed inside the glass valve of flashtube 7 for light irradiation treatment and prevention. In this instance, the measurements of the energy of the light in the wavelength range between 600 nm and 700 nm were made by using Model MPCD-3000 (lighting voltage of 200 V, and main capacitor capacitance of 350 μF) manufactured by Otsuka Electronics Co., Ltd.

Energies of the light in μW/cm² were measured twice, for instance, and an average value of the measurements was obtained with a light receiver of the above measuring instrument set at a distance of 400 mm from flashtube 7 for light irradiation treatment and prevention. The obtained average value of the light energy is converted as a relative energy ratio with respect to a 100% light energy (μW/cm²) of the xenon discharge tube in the wavelength range between 600 nm and 700 nm inclusive, and tabulated in Table 2.

It is known, as shown in FIG. 4 and Table 2, that the relative energy ratio of flashtube 7 for light irradiation treatment and prevention can be maintained at 90% or more when the mixing ratio of xenon gas is 1.21 mol % or less relative to 100 mol % of the mixed gas. It is also known that the relative energy ratio of flashtube 7 for light irradiation treatment and prevention can be maintained at 95% or even higher if the mixing ratio of xenon gas is 0.44 mol % or less relative to 100 mol % of the mixed gas.

TABLE 2

| Mixing Ratio of Xenon Gas | Relative Energy Ratio |
|---|---|
| 0.00 mol % | 95.8% |
| 0.23 mol % | 96.9% |
| 0.44 mol % | 96.6% |
| 1.21 mol % | 92.4% |
| 2.34 mol % | 87.8% |

The present inventor has confirmed that there is a proportional relationship between a filled-gas pressure and the lighting voltage for both the conventional neon discharge tube in which neon gas is enclosed, and flashtube 7 for light irradiation treatment and prevention of the present invention. In other words, it is presumed according to the above proportional relationship that the advantageous effects of maintaining the amount of light and reducing the lighting voltage attributed to the mixture of xenon gas and neon gas can be ensured as they are, even when the filled-gas pressure is changed.

Description is provided next of operation and function of light irradiation treatment and prevention device 1 according to this exemplary embodiment of the invention with reference to FIG. 2.

First, a specific part of a living body such as a hand is inserted in light irradiating section 15 shown in FIG. 2, and an electric power to light irradiation treatment and prevention device 1 is turned on. This causes flashtube 7 for light irradiation treatment and prevention, which is the light source inside light-emitting unit 2, to emit and radiate light in the range of 600 nm to 700 nm inclusive.

A part of the light radiated from light-emitting unit 2 and reflected by reflector 8 travels via first reflecting member 11 of reflection frame 3, and irradiates an upper area of the specific part of the subject of irradiation through first transparent surface 10 of transparent frame 4. On the other hand, a remaining part of the light radiated from light-emitting unit 2 and reflected by reflector 8 irradiates a lower area of the specific part of the subject of irradiation through second transparent surface 12 of transparent frame 4.

In this case, flashtube 7 for light irradiation treatment and prevention of this exemplary embodiment emits the light having a peak wavelength in a range from 600 nm to 700 nm inclusive. It is therefore unnecessary to use an expensive band-pass filter.

Moreover, flashtube 7 for light irradiation treatment and prevention of this exemplary embodiment lights up with the lighting voltage of 200 V or less. At the same time, flashtube 7 for light irradiation treatment and prevention radiates the light having 90% or more energy in comparison to a value when the light radiated from a xenon discharge tube has 100% energy. Accordingly, it becomes possible to make flashtube 7 for light irradiation treatment and prevention irradiate the subject of irradiation with energy of the light having wavelengths between 600 nm and 700 nm inclusive that are necessary for the light irradiation treatment and prevention without increasing the lighting voltage.

It should be understood that the light irradiation treatment and prevention device of the present invention is not limited to only the exemplary embodiment described above such that various changes and modifications may be made within the scope and without departing from the spirit of the invention.

In light irradiation treatment and prevention device 1 of the above exemplary embodiment, the description is provided of an example that irradiates a hand with the light radiated from flashtube 7 for light irradiation treatment and prevention, however, this is not restrictive. For example, the irradiation may be made on any other part of the living body such as a part where the user wants to suppress and prevent production of pro-inflammatory cytokine or a part affected by a disease. Specifically, the other parts of the living body to be irradiated include a shoulder, a lower back, a foot, and the whole body, or any area of the body may be irradiated. In addition, the subject of irradiation is not limited to humans, but specific parts of other living bodies like animals other than humans can be irradiated for treatment. When such is the case, the structure may be altered or changed as appropriate to fit for a specific part of such a living body to be irradiated without limiting the structure to that of light irradiation treatment and prevention device 1 in this exemplary embodiment.

As described above, the present invention discloses a flashtube for light irradiation treatment and prevention used for medical treatment or prevention by irradiating a specific part of a living body with light. The flashtube includes a glass valve, and filler gas contained in the glass valve. The filler gas is composed of mixed gas of neon gas and xenon gas, and the composition includes the xenon gas ranging from 0.23 mol % to 1.21 mol % inclusive in a ratio relative to 100 mol % of the mixed gas.

It becomes possible by virtue of this composition to keep the lighting voltage of 200 V or less to light up the flashtube and to ensure 90% or more energy of the light radiated from the flashtube as compared to a value when the light radiated from the xenon discharge tube has 100% of energy. Thus achieved is the flashtube for light irradiation treatment and prevention that can produce energy of the light having wavelengths between 600 nm and 700 nm inclusive that are necessary for the light irradiation treatment and prevention without increasing the lighting voltage.

Alternatively, the flashtube for light irradiation treatment and prevention of the present invention may have a structure in which the glass valve is filled with filler gas having xenon gas in a mixing ratio ranging from 0.23 mol % to 0.44 mol % inclusive relative to 100 mol % of the mixed gas.

With the above structure, energy of the light radiated from the flashtube for light irradiation treatment and prevention can be brought to 95% or more as compared to the value when the light radiated from the xenon discharge tube has 100% of energy. As a result, the energy of the light having wavelengths in the range between 600 nm and 700 nm inclusive that is necessary for the light irradiation treatment and prevention can be obtained more efficiently without increasing the lighting voltage.

In addition, the present invention discloses a light irradiation treatment and prevention device for medical treatment or prevention by irradiating a specific part of a living body with the light radiated from the above-described flashtube for light irradiation treatment and prevention. The device may be configured to irradiate the specific part of the living body with the light having wavelengths in the range between 600 nm and 700 nm inclusive, radiated from the flashtube for light irradiation treatment and prevention.

According to this structure, energy of the light radiated from the flashtube for light irradiation treatment and prevention can be brought to 95% or more as compared to the value when the light radiated from the xenon discharge tube has 100% of energy, without increasing the lighting voltage. This makes an expensive band-pass filter unnecessary. As a result, what can be achieved is the light irradiation treatment and prevention device of a low cost, yet capable of radiating energy of the light having the wavelengths ranging between 600 nm to 700 nm inclusive that are necessary for the light irradiation treatment and prevention.

### INDUSTRIAL APPLICABILITY

The present invention is applicable to such usages as flashtubes for light irradiation treatment and prevention, light irradiation treatment and prevention devices and the like, that are required to have low-cost structures, and produce energy of the light having wavelengths ranging from 600 nm to 700 nm inclusive necessary for the light irradiation treatment and prevention without increasing lighting voltages of the flashtubes.

### REFERENCE MARKS IN THE DRAWINGS

1 light irradiation treatment and prevention device
2 light-emitting unit
3 reflection frame
4 transparent frame
5 light-emission controller
6 enclosure
7 light source (flashtube for light irradiation treatment and prevention)
8 reflector
9 protective panel
10 first transparent surface
11 first reflecting member
12 second transparent surface
13 second reflecting member
14 third reflecting member
15 light irradiating section
16 casing

1. A flashtube for light irradiation treatment and prevention used for medical treatment or prevention by irradiating a specific part of a living body with light, the flashtube comprising:
a glass valve; and
filler gas contained inside the glass valve,
wherein the filler gas comprises mixed gas of at least neon gas and xenon gas, and includes the xenon gas in a mixing ratio between 0.23 mol % and 1.21 mol % inclusive relative to 100 mol % of the mixed gas.

2. The flashtube for light irradiation treatment and prevention according to claim 1, wherein the glass valve is filled with the filler gas that includes the xenon gas in a mixing ratio between 0.23 mol % and 0.44 mol % inclusive relative to 100 mol % of the mixed gas.

3. A light irradiation treatment and prevention device for medical treatment or prevention by irradiating a specific part of a living body with light radiated from the flashtube as defined in claim 1, wherein the specific part of the living body is irradiated with the light of a wavelength in a range between 600 nm and 700 nm inclusive from the flashtube.

4. A light irradiation treatment and prevention device for medical treatment or prevention by irradiating a specific part of a living body with light radiated from the flashtube as defined in claim 2, wherein the specific part of the living body is irradiated with the light of a wavelength in a range between 600 nm and 700 nm inclusive from the flashtube.
